(19) United States
(12) Patent Application Publication
(10) Pub. No.: US 2004/0162432 A1
(43) Pub. Date: Aug. 19, 2004

(54) Substituted octahydrophenanthrene compounds and use thereof as NMDA antagonists

(76) Inventors: Helmut Buschmann, Esplugues de Llobregat (ES); Werner Englberger, Stolberg (DE); Michael Przewosny, Aachen (DE); Hans Schick, Berlin (DE); Birgitta Henkel, Berlin (DE); Michael Sattlegger, Bonn (DE)

Correspondence Address: Geza C. Ziegler, PERMAN & GREEN, LLP, 425 Post Road, Fairfield, CT, 06824, US

(21) Appl. No.: 10/778,380

(22) Filed: Feb. 13, 2004

Related U.S. Application Data
(63) Continuation of application No. PCT/EP02/08866, filed on Aug. 8, 2002.

(30) Foreign Application Priority Data
Aug. 16, 2001 (DE) 101 40 213.9

(51) Int. Cl. C07D 211/06

(52) U.S. Cl. 546/204; 558/418; 564/86; 564/384; 548/528

(57) ABSTRACT

The invention relates to substituted octahydrophenanthrene compounds of general formula (I), to a method for their production, to medicaments containing these compounds and to the use of said compounds for producing medicaments.

The present invention relates to substituted octahydrophenanthrene compounds, a process for the production thereof, pharmaceutical preparations containing these compounds and the use of these compounds for the production of pharmaceutical preparations.

The treatment of chronic and non-chronic pain is of great significance in medicine. There is a worldwide requirement for effective therapeutic methods for providing tailored and targeted treatment of chronic and non-chronic pain, this being taken to mean pain treatment which is effective and satisfactory from the patient's standpoint. This is clear from the large number of scientific papers relating to applied analgesia or to basic nociception research which have recently been published.

Conventional opioids, such as morphine for example, are highly effective in treating severe to extreme pain. However, the use thereof is limited by known side-effects such as respiratory depression, vomiting, sedation, constipation and development of tolerance. Moreover, they are less effective in treating neuropathic or incidental pain, which is in particular experienced by tumour patients.

Opioids exert their analgesic effect by binding to membrane receptors belonging to the family of G protein-coupled receptors. The biochemical and pharmacological characterisation of subtypes of these receptors has prompted hopes that corresponding subtype-specific active ingredients may have an effect/side-effect profile which differs from that of, for example, morphine. Further pharmacological investigations have now tentatively revealed the existence of various subtypes of these opioid receptors.

There are moreover further receptors and ion channels which play a substantial role in the system governing the genesis and transmission of pain. The NMDA ion channel, through which a substantial part of synaptic communication passes, is of particular significance in this connection. This channel controls the exchange of calcium ions between a neuronal cell and its environment.

In the unactivated state, the NMDA ion channels are in each case closed by individual magnesium ions which are located within the channel and cannot pass therethrough due to their size. In the activated state, the smaller calcium and sodium ions are able to pass through the channel. The (+)-MK801 binding site of the NMDA ion channel is likewise located within this membrane protein. Substances such as MK801 exhibit an affinity with this binding site and close the NMDA ion channel.

The development of the patch-clamp technique has made it possible to elucidate the physiological significance of ion channel-selective substances. It has thus been possible clearly to demonstrate the effect of NMDA antagonists on the influence of calcium ions within the cell. It has also been established that these substances themselves have their own antinociceptive potential, such as for example ketamine. One important fact is that their mode of action differs greatly from that of, for example, opiates, as NMDA antagonists act directly on the cell's calcium balance, which vitally determines the transmission of pain. The possibility of also treating neuropathic types of pain successfully thus arises.

Various NMDA antagonists from the group comprising tetrahydroquinoline derivatives have already been described in J. Med. Chem. (1992) 35, pages 1954-1968; J. Med. Chem. (1992) 35, pages 1942-1953 and Med. Chem. Res. (1991) 1; pages 64-73 and in EP 0 386 839, WO 97/12879, WO 98/07704 and WO 98/42673.

Numerous indications which are susceptible to treatment with NMDA antagonists are furthermore stated, inter alia also including the treatment of pain.

There is, however, still a requirement for effective NMDA antagonists which are preferably suitable for combatting pain.

One object underlying the present invention was accordingly to provide novel compounds which are in particular suitable as pharmaceutical active ingredients in pharmaceutical preparations, preferably as pharmaceutical preparations for combatting pain, in particular for the treatment of chronic or neuropathic pain. These active ingredients should moreover also be suitable for the treatment or prevention of neurodegenerative diseases, in particular of Alzheimer's disease, Parkinson's disease or Huntington's chorea, of migraine, stroke, cerebral ischaemia, cerebral infarct, cerebral oedema, schizophrenia, psychoses brought about by elevated amino acid levels, AIDS dementia, Tourette's syndrome, inflammatory and/or allergic reactions, insufficiency states of the central nervous system, especially hypoxia and anoxia, perinatal asphyxia, depression, mental health conditions, epilepsy, urinary incontinence, pruritus, tinnitus, diarrhoea, for anxiolysis or for anaesthesia.

It has surprisingly been found that substituted octahydrophenanthrene compounds of the general formula I below act as NMDA antagonists. The compounds according to the invention surprisingly exhibit an affinity with the (+)-MK801 binding site of the NMDA ion channel. They are accordingly suitable for combatting pain, in particular for combatting chronic or neuropathic pain, but also for the treatment or prevention of neurodegenerative diseases, in particular of Alzheimer's disease, Parkinson's disease or Huntington's chorea, of migraine, stroke, cerebral ischaemia, cerebral infarct, cerebral oedema, schizophrenia, psychoses brought about by elevated amino acid levels, AIDS dementia, Tourette's syndrome, inflammatory and/or allergic reactions, insufficiency states of the central nervous system, especially hypoxia and anoxia, perinatal asphyxia, depression, mental health conditions, epilepsy, urinary incontinence, pruritus, tinnitus, diarrhoea, for anxiolysis or for anaesthesia.

The present invention accordingly provides substituted octahydrophenanthrene compounds of the general formula I

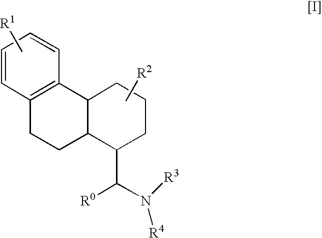

in which

R⁰, R¹ and R², identical or different, denote hydrogen, a linear or branched, saturated or unsaturated C₁-C₁₂ aliphatic residue, a cycloaliphatic saturated or unsaturated C₃-C₇ residue, an aryl or heteroaryl residue optionally attached via a C₁-C₃ alkylene residue, a halogen or a group of the formula —CN, —OR⁵, —SR⁵, —CHF₂, —CE₃, —NHR⁵, —N(R⁵)₂, —NO₂, —SO₂R⁵, or R² denotes an oxo residue,

R³ and R⁴, identical or different, denote hydrogen, a linear or branched, saturated or unsaturated C₁-C₁₂ aliphatic residue, a cycloaliphatic saturated or unsaturated C₃-C₇ residue, an aryl or heteroaryl residue optionally attached via a C₁-C₃ alkylene residue or R³ and R⁴ together form a (CH₂)₂₋₇ ring and

R⁵ denotes a linear or branched, saturated or unsaturated C₁-C₁₂ aliphatic or a C₃-C₇ cycloaliphatic residue, an aryl or heteroaryl residue, in the form of the racemates, diastereomers or enantiomers thereof and in the form of corresponding bases or of a corresponding physiologically acceptable salt.

Preferred substituted octahydrophenanthrene compounds of the general formula I are those in which the residue R¹ denotes hydrogen, a C₁-C₆ alkyl residue, a methoxy, hydroxyl, benzyl or phenethyl residue or a halogen, preferably chlorine or fluorine.

Further preferred substituted octahydrophenanthrene compounds of the general formula I are those in which the residue R² denotes a C₁-C₆ alkyl residue, a benzyl or phenethyl residue, preferably a tert-butyl residue.

Preferred substituted octahydrophenanthrene compounds of the general formula I are also those in which the residue R³ and/or R⁴ denotes a C₁-C₃ alkyl residue, particularly preferably those in which each denotes a methyl residue.

Further preferred substituted octahydrophenanthrene compounds of the general formula I are those in which the residue R⁵ denotes a C₁-C₃ alkyl residue, particularly preferably a methyl residue.

The aliphatic residues may be mono- or polysubstituted. If the aliphatic residues comprise more than one substituent, these may be identical or different and be attached both to the same and to different atoms of the aliphatic residue. The aliphatic residue is preferably selected from the group consisting of optionally at least mono-substituted methyl, ethyl, propyl, isopropyl, n-butyl, sec.-butyl, tert-butyl, n-pentyl, neopentyl, n-hexyl, n-heptyl, n-octyl, n-nonyl, n-decyl, propenyl, butenyl, pentenyl, hexenyl, heptenyl, octenyl, propynyl, butynyl, pentynyl, hexynyl, heptynyl and octynyl. The substituents are preferably selected from the group consisting of F, Cl, Br, I, NH₂, SH and OH.

The cycloaliphatic residues may be mono- or polysubstituted and/or optionally unsaturated or saturated. If the cycloaliphatic residues comprise more than one substituent, these may be identical or different and be attached both to the same and to different atoms of the cycloaliphatic residue. A preferred cycloaliphatic residue is an optionally at least monosubstituted, saturated or unsaturated cyclopropyl, cyclobutyl, cyclopentyl, cyclohexyl, cycloheptyl, cyclopentenyl, cyclohexenyl or cycloheptenyl residue. The substituents are preferably selected from the group consisting of halogen, NH₂, SH and OH.

For the purposes of the present invention, an aryl residue is also taken to mean those aromatic hydrocarbon residues which are fused with a saturated or at least monounsaturated hydrocarbon ring system.

A preferred aryl residue is an optionally mono- or polysubstituted phenyl, naphthyl or anthracenyl residue, particularly preferably an optionally monosubstituted phenyl residue.

If the aryl residue comprises more than one substituent, these may be identical or different. The substituents are preferably selected from the group consisting of halogen, NH₂, SH, OH, CF₃, CN, NO₂, OR⁵, SR⁵, NR⁵R⁵ and a C₁₋₆ alkyl, C₁₋₆ alkoxy, C₂₋₈ alkenyl, C₂₋₈ alkynyl, phenyl, phenoxy or benzyloxy residue which is unsubstituted or at least monosubstituted with a halogen, NH₂, SH, OH, CF₃, CN or NO₂, wherein R⁵ has the meaning already stated above.

For the purposes of the present invention, a heteroaryl residue is understood to mean also those heteroaromatic, hydrocarbon residues which are fused with a saturated or at least monounsaturated hydrocarbon ring system. The heteroaryl residue preferably contains a heteroatom selected from the group consisting of sulfur, nitrogen and oxygen.

The heteroaryl residue is preferably an optionally at least mono-substituted thiophenyl, furanyl, pyrrolyl, pyridinyl, pyrimidinyl, quinolinyl, isoquinolinyl, phthalazinyl or quinazolinyl residue. If the heteroaryl residue comprises more than one substituent, these may be identical or different. The substituents are preferably selected from the group consisting of F, Cl, Br, I, NH₂, SH, OH, CF₃, CN, NO₂, OR⁵, SR⁵, NR⁵R⁵ and a C₁₋₆ alkyl, C₁₋₆ alkoxy, C₂₋₈ alkenyl, C₂₋₈ alkynyl, phenyl, phenoxy or benzyloxy residue which is unsubstituted or at least monosubstituted with F, Cl, Br, I, NH₂, SH, OH, CF₃, CN or NO₂, wherein the residue R⁵ has the above-stated meaning.

The following substituted octahydrophenanthrene compounds are very particularly preferred:

8-dimethylaminomethyl-4b,5,6,7,8,8a,9,10-octahydrophenanthrene,

8-dimethylaminomethyl-3-methoxy-4b,5,6,7,8,8a,9,10-octahydrophenanthrene,

8-dimethylaminomethyl-4b,5,6,7,8,8a,9,10-octahydrophenanthren-3-ol,

8-dimethylaminomethyl-2-fluoro-4b,5,6,7,8,8a,9,10-octahydrophenanthrene,

8-dimethylaminomethyl-3-chloro-4b,5,6,7,8,8a,9,10-octahydrophenanthrene, and

6-tert-butyl-8-dimethylaminomethyl-4b,5,6,7,8,8a,9,10-octahydrophenanthrene,

and a corresponding physiologically acceptable salt, preferably a hydrochloride.

The present invention also provides a process for the production of the substituted octahydrophenanthrene compounds according to the invention, in which

a compound of the general formula II is converted,

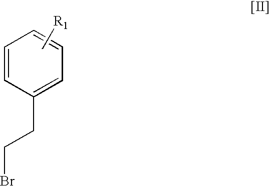

with Mg in a suitable organic solvent, preferably in an organic solvent containing ether, particularly preferably in diethyl ether or tetrahydrofuran, and optionally under a protective gas atmosphere, preferably under argon gas, into the corresponding Grignard compound, then the reaction mixture is cooled, preferably to 10 to 30° C., and this reaction mixture is reacted with a compound of the general formula III,

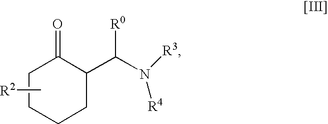

which is optionally dissolved in an organic solvent and the resultant compound IV

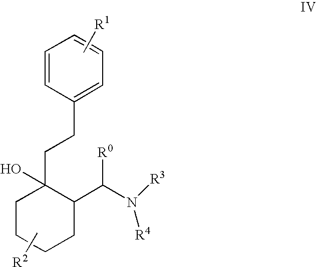

is optionally purified and/or isolated using conventional methods,

the compound IV is reacted by heating with a suitable acid, preferably HBr or formic acid, in a suitable solvent, preferably water or formic acid, is neutralised with a suitable base and is optionally purified and/or isolated using conventional methods,

in which the residues R⁰-R⁵ have the meaning according to claim 1.

Compounds of the general formulae II and III are commercially obtainable or may straightforwardly be produced from commercial precursors using production processes known to the person skilled in the art.

The general reaction mechanism of the reaction of the compounds of the general formulae II and III is familiar to the person skilled in the art in the field of organic synthesis. The person skilled in the art knows the reaction of a compound III where R⁰=H with a compound II as a “Mannich reaction” and the reaction of a compound III where R⁰≠H as a “Risch-variant Mannich reaction”.

The quantities to be used of the reaction components of the general formulae II, III and IV, of the magnesium and of the suitable acids and bases, the temperature during the reaction and the duration of the reaction may vary. The suitable quantity of the components to be used for the particular reaction, the suitable temperature and the suitable duration of the reaction may be determined by the person skilled in the art by simple preliminary testing.

The reaction of the compounds of the general formulae II and III to yield a compound of the general formula IV preferably lasts 0.1 to 5 hours, particularly preferably 0.5 to 2 hours.

The temperature during the reaction of the compound of the general formula IV with a suitable acid is preferably 50 to 150° C., particularly preferably 80 to 120° C.

It is furthermore preferred for the reaction of the compound of the general formula IV with a suitable acid to proceed over a period of 1 to 8 hours, particularly preferably over 3 to 6 hours.

The reaction of a compound of the general formula I to yield a corresponding hydrochloride preferably proceeds with trimethylchlorosilane in a suitable solvent, preferably ethyl methyl ketone.

The particular reaction components of the general formulae II, III and metallic magnesium, suitable acids and bases may be purchased commercially or be produced using conventional methods known to the person skilled in the art.

The substituted octahydrophenanthrene compounds according to the invention of the general formula I may be isolated in accordance with the process according to the invention as a free base or as a salt. The free base of the respective compound according to the invention of the general formula I may be converted into the corresponding physiologically acceptable salt using conventional methods known to the person skilled in the art, for example by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid.

The free base of the respective compound of the general formula I according to the invention may also be converted into the corresponding physiologically acceptable salt with the free acid or a salt of a sugar substitute, such as for example saccharin, cyclamate or acesulfame.

Conversion of the free base of the respective compound according to the invention of the general formula I into the corresponding hydrochloride may preferably also be obtained by combining the compound of the general formula I according to the invention, dissolved in a suitable organic solvent, such as for example butan-2-one (methyl ethyl ketone), as a free base with trimethylsilyl chloride (TMSCl).

The reaction of a compound of the general formula I with trimethylchlorosilane to yield a corresponding hydrochloride preferably proceeds with stirring over a period of 2 to 8 hours, particularly preferably over 3 to 6 hours.

The temperature of this reaction is preferably 0 to 50° C., particularly preferably 20 to 30° C.

If the substituted octahydrophenanthrene compounds of the general formula I according to the invention are obtained by the production process according to the invention in the form of the racemates thereof or other mixtures of their various enantiomers and/or diastereomers, these may, if necessary, be separated and optionally isolated by conventional processes known to the person skilled in the art. Examples are chromatographic separation processes, in particular liquid chromatography processes at standard pressure or at elevated pressure, preferably MPLC and HPLC methods, and fractional crystallisation processes. Individual enantiomers, e.g. diastereomeric salts formed by means of HPLC on a chiral phase or by means of crystallisation with chiral acids, such as (+)-tartaric acid, (−)-tartaric acid or (+)-10-camphorsulfonic acid, may here in particular be separated from one another.

The substituted octahydrophenanthrene compounds of the general formula I according to the invention are suitable as pharmaceutical active ingredients in pharmaceutical preparations.

The present invention therefore also provides pharmaceutical preparations which contain at least one substituted octahydrophenanthrene compound of the general formula I according to the invention and optionally physiologically acceptable auxiliary substances.

The pharmaceutical preparations according to the invention are preferably suitable for combatting pain, particularly preferably for combatting chronic or neuropathic pain.

The pharmaceutical preparations according to the invention are also suitable for the treatment or prevention of neurodegenerative diseases, in particular of Alzheimer's disease, Parkinson's disease or Huntington's chorea or for the treatment or prevention of migraine, stroke, cerebral ischaemia, cerebral infarct, cerebral oedema, schizophrenia, psychoses brought about by elevated amino acid levels, AIDS dementia, Tourette's syndrome, inflammatory and/or allergic reactions, insufficiency states of the central nervous system, especially hypoxia and anoxia, perinatal asphyxia, depression, mental health conditions, epilepsy, urinary incontinence, pruritus, tinnitus, diarrhoea, for anxiolysis or for anaesthesia.

The use of at least one substituted octahydrophenanthrene compound of the general formula I for the production of a pharmaceutical preparation for combatting pain,

in particular for combatting chronic or neuropathic pain, for the treatment or prevention of neurodegenerative diseases, in particular of Alzheimer's disease, Parkinson's disease or Huntington's chorea or for the treatment or prevention of migraine, stroke, cerebral ischaemia, cerebral infarct, cerebral oedema, schizophrenia, psychoses brought about by elevated amino acid levels,

AIDS dementia, Tourette's syndrome, inflammatory and/or allergic reactions, insufficiency states of the central nervous system, especially hypoxia and anoxia, perinatal asphyxia, depression, mental health conditions, epilepsy, urinary incontinence, pruritus, tinnitus, diarrhoea, for anxiolysis or anaesthesia is accordingly also provided by the present invention.

The pharmaceutical preparations according to the invention may also contain mixtures of various stereoisomers of one or more octahydrophenanthrene compounds according to the invention. Various enantiomers of an octahydrophenanthrene compound according to the invention may accordingly, for example, also be present in non-equimolar quantities.

In addition to at least one substituted octahydrophenanthrene compound according to the invention, the pharmaceutical preparations according to the invention conventionally contain further physiologically acceptable auxiliary substances, which are preferably selected from the group consisting of matrix materials, fillers, solvents, diluents, dyes and binders. The pharmaceutical preparations according to the invention may be present as liquid, semisolid or solid dosage forms, for example in the form of solutions for injection, drops, succi, syrups, sprays, suspensions, tablets, patches, capsules, transdermal delivery systems, suppositories, ointments, creams, lotions, gels, emulsions, aerosols or in multiparticulate form, for example in the form of pellets or granules, and also be administered as such.

Selection of the physiologically acceptable auxiliary substances and the quantities thereof which are to be used depends upon whether the pharmaceutical preparation is to be administered orally, perorally, parenterally, intravenously, intraperitoneally, intradermally, intramuscularly, intranasally, buccally, rectally or topically, for example onto infections of the skin, mucous membranes or eyes. Preparations in the form of tablets, coated tablets, capsules, granules, drops, succi and syrups are suitable for oral administration, while solutions, suspensions, easily reconstitutible dried preparations and sprays are suitable for parenteral, topical and inhalatory administration. Substituted octahydrophenanthrene compounds according to the invention in a depot in dissolved form or in a dressing, optionally with the addition of skin penetration promoters, are suitable percutaneous administration preparations. Orally or percutaneously administrable formulations may release the substituted octahydrophenanthrene compounds according to the invention in delayed manner.

Production of the pharmaceutical preparations according to the invention proceeds with the assistance of conventional means, devices, methods and processes known to the person skilled in the art, such as are described for example in “Remington's Pharmaceutical Sciences”, ed. A. R. Gennaro, 17th ed., Mack Publishing Company, Easton, Pa. (1985), in particular in part 8, chapters 76 to 93. The corresponding literature description is hereby introduced as a reference and is deemed to be part of the disclosure.

The quantity of the respective compound of the general formula I according to the invention to be administered to the patient may vary and is for example dependent on the weight or age of the patient and on the mode of administration, the indication and the severity of the complaint. 0.5 to 500 mg per kg patient body weight of at least one compound of the general formula I according to the invention are conventionally administered.

The present invention is explained below with reference to Examples. These explanations are given merely by way of example and do not restrict the general concept of the invention.

### EXAMPLES

The chemicals and solvents used for the production of the octahydrophenanthrene compounds according to the invention were purchased commercially, for example from Acros, Avocado, Aldrich, Fluka, Lancaster, Maybridge, Merck, Sigma or TCI, or produced using conventional methods known to the person skilled in the art.

Thin-layer chromatography was performed with pre-coated silica gel 60 F 254 HPLC plates from E. Merck, Darmstadt.

The yields of the compounds produced have not been optimised.

All melting temperatures are uncorrected.

Analysis was performed by ESI mass spectroscopy or NMR spectroscopy.

Unless stated otherwise, petroleum ether with the boiling range of 50-70° C. was used. The term “ether” denotes diethyl ether.

Silica gel 60 (0.040-0.063 mm) from E. Merck, Darmstadt, was used as the stationary phase for the column chromatography.

### Example 1

### 8-Dimethylaminomethyl-4b,5,6,7,8,8a,9,10-octahydrophenanthrene hydrochloride, compound (1)

1st stage

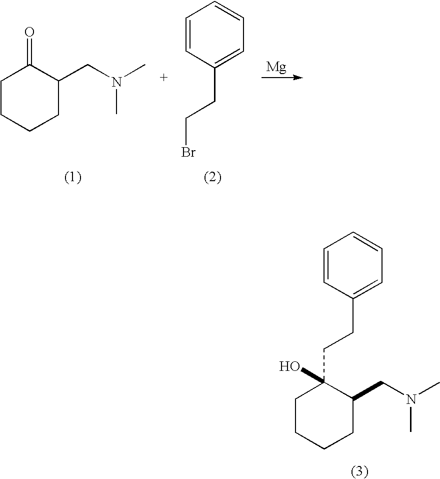

Starting from magnesium (128 mmol) and phenethyl bromide (2) (128 mmol) under argon, the Grignard reaction was initiated in diethyl ether (15 ml) by first of all adding 20% of the reagent (2). While boiling, the remaining 80% of the reagent (2) were added within 30 minutes in diethyl ether (40 ml). After boiling for 1 hour, the temperature was reduced to 0C and the ketone (1) (32 mmol), dissolved in diethyl ether (10 ml), was added within 10 minutes. After stirring for 1 hour at room temperature, working up was performed by introducing the reaction mixture into 5N HCl (80 ml, 0.4 mol) with ice cooling and removing all nonpolar constituents by extraction with diethyl ether. The tertiary alcohol (3) (60 mmol) was isolated from the aqueous phase, which had been alkalised with 5N sodium hydroxide solution, by extraction with diethyl ether.

2nd stage

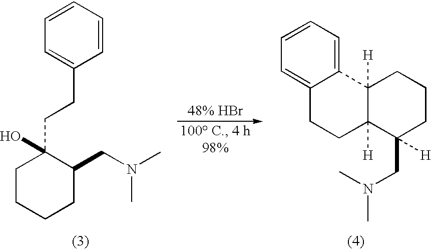

The alcohol (3) (1.5 g, 5.7 mmol) was heated to 100° C. for 4 h with 48% HBr (17 ml, 136 mmol). After neutralisation with sodium hydroxide solution (pH 8-9) and extraction by shaking with diethyl ether, the phenanthrene (4) was obtained as a diastereomerically pure yellow oil in a yield of 98%.

3rd stage

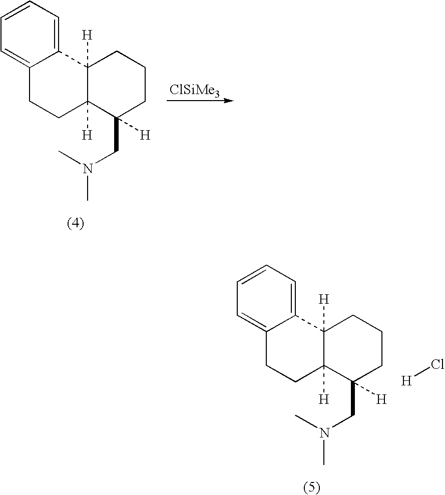

The phenanthrene (4) (0.8 g, 3.4 mmol) was dissolved in ethyl methyl ketone (4 ml), combined with trimethylchlorosilane (0.64 ml, 5.1 mmol) and stirred for 4.5 h at room temperature. The hydrochloride (5) was isolated as a white solid with a yield of 66% and a melting point of 225-229° C.

### Example 2

### 8-Dimethylaminomethyl-3-methoxy-4b,5,6,7,8,8a,9,10-octahydrophenanthrene hydrochloride, compound (2)

The first step of the preparation of compound (2) was performed in a similar manner to Example 1. 4-Methoxyphenethyl bromide was used instead of phenethyl bromide. After the Grignard reaction, cyclisation of (6) was performed with formic acid.

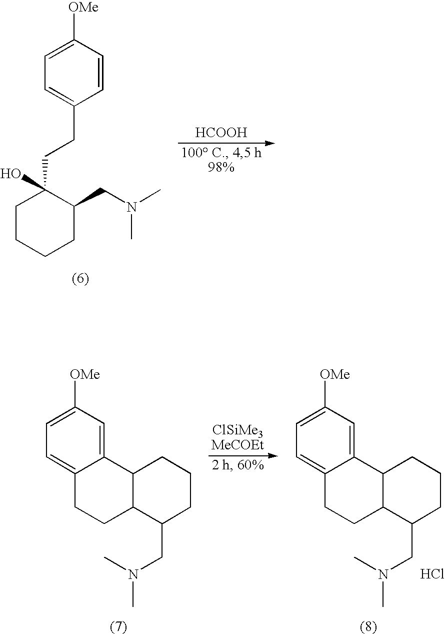

The phenanthrene was synthesised by heating the alcohol (6) (0.45 g, 1.64 mmol) with formic acid (1 ml) to 100° C. for 4.5 h. After alkalisation with sodium hydroxide solution and extraction with diethyl ether, the free base (7) of the phenanthrene was obtained. ClSiMe₃ (0.26 ml, 2.08 mmol) was then added to a solution of the phenanthrene (7) (0.38 g, 1.38 mmol) in MeCOEt (20 ml). After 2 h, the hydrochloride was obtained as a white solid. Compound (8) was isolated with a yield of 60% and a melting range of 220-223° C.

### Example 3

### 8-Dimethylaminomethyl-4b,5,6,7,8,8a,9,10-octahydrophenanthren-3-ol hydrochloride, compound (3)

The compound was prepared in a manner similar to Example 1. 4-Methoxyphenethyl bromide was used instead of phenethyl bromide. After precipitation as the hydrochloride, the compound (3) was obtained as a white solid with a melting range of 250-253° C. Yield was 265 mg.

### Example 4

### 8-Dimethylaminomethyl-2-fluoro-4b,5,6,7,8,8a,9,10-octahydrophenanthrene hydrochloride, compound (4)

The compound was prepared in a manner similar to Example 1. 3-Fluorophenethyl bromide was used instead of phenethyl bromide. After precipitation as the hydrochloride, the compound 4 was obtained as a white solid which decomposes from 270° C. Yield was 113 mg.

### Example 5

### 8-Dimethylaminomethyl-3-chloro-4b,5,6,7,8,8a,9,10-octahydrophenanthrene hydrochloride, compound (5)

The compound was prepared in a manner similar to Example 1. 4-Chlorophenethyl bromide was used instead of phenethyl bromide. After precipitation as the hydrochloride, the compound (5) was obtained as a white solid with a melting point of 240-243° C. Yield was 343 mg.

### Example 6

### 6-tert-Butyl-8-dimethylaminomethyl-4b,5,6,7,8,8a,9,10-octahydrophenanthrene hydrochloride, compound (6)

The compound was prepared in a manner similar to Example 1. 4-tert-Butyl-2-dimethylaminomethylcyclohexanone was used instead of 2-dimethylaminohexanone.

After precipitation as the hydrochloride, compound (6) was obtained as a colourless solid which has a melting range of 267-270° C. Yield was 301 mg.

Molecular Pharmacological Investigations:

The investigations for determining the affinity of the octahydrophenanthrene compounds of the general formula I according to the invention with the (+)-MK801 binding site of the ionotropic NMDA receptor was performed on brain membrane homogenates (homogenate of rat brain without the cerebellum, pons and medulla oblongata of male rats, Wistar strain, Charles River, WIGA GmbH, Sulzbach, Germany) as described in B. M. Baron et al., Journal of Pharmacology and Experimental Therapeutics, vol. 279, pages 62-68, 1996. The corresponding literature description is hereby introduced as a reference and is deemed to be part of the disclosure.

To this end, after separation of the cerebellum, pons and medulla oblongata, freshly prepared rat brains were disrupted for 1 minute with a Polytron homogeniser at 6,000 rpm in 50 mmol/l of tris/HCl (pH 7.7) with ice cooling and were then centrifuged for 15 min at 4° C. and 60,000 g. After decanting off and discarding the supernatant, resuspending the membrane pellet in 50 mmol/l of tris/HCl (pH 7.7) and disrupting it with a homogeniser at 2,000 rpm for 1 minute, centrifuging was again performed for 15 min at 4° C. and 60,000 g. The supernatant was again discarded and the membrane pellet was homogenised in 50 mmol/l of tris/HCl (pH 7.7) (2,000 rpm for 1 minute) and frozen in aliquots at −70° C.

Aliquots were in each case thawed for the receptor binding test and then centrifuged for 15 min at 4° C. and 60,000 g. After decanting off and discarding the supernatant, the membrane pellet was suspended for the binding test with binding test buffer and homogenised (2,000 rpm for 1 minute).

The buffer used for the binding test was a buffer of 5 mmol/l of tris/HCl (pH 7.7), supplemented with 30 μmol/l of glycine and 100 μmol/l of glutamic acid.

The radioactively labelled ligand used was 1 nmol/μl of (³H)-(+)-MK801 ((5R,10S)-(+)-5-methyl-10,11-dihydro-5H-dibenzo(a,d)cyclohepten-5,10-imine). The level of non-specific binding was determined in the presence of 10 μmol/l of non-radioactively labelled (+)-MK801.

In further batches, the compounds according to the invention from the above-stated Examples

(1) 8-dimethylaminomethyl-4b,5,6,7,8,8a,9,10-octahydrophenanthrene,

(2) 8-dimethylaminomethyl-3-methoxy-4b,5,6,7,8,8a,9,10-octahydrophenanthrene,

(3) 8-dimethylaminomethyl-4b,5,6,7,8,8a,9,10-octahydrophenanthren-3-ol,

(4) 8-dimethylaminomethyl-2-fluoro-4b,5,6,7,8,8a,9,10-octahydrophenanthrene,

(5) 8-dimethylaminomethyl-3-chloro-4b,5,6,7,8,8a,9,10-octahydrophenanthrene,

(6) 6-tert-butyl-8-dimethylaminomethyl-4b,5,6,7,8,8a,9,10-octahydrophenanthrene,

were added in concentration series and the displacement of the radioactive ligand from its specific binding on the NMDA receptor was determined. Each of the triplicate batches was incubated for 40 minutes at 25° C. and then harvested by filtration through glass fibre filters (GF/B) in order to determine the radioactive ligand bound to the brain membrane homogenate. The radioactivity retained on the glass fibre filter disk was measured, after addition of scintillating liquid, in the β-counter.

The percentage inhibition of the specific binding of the ligand (³H)-(+)-MK801 obtained from triplicate batches in the presence of in each case 10 μmol/l of the substituted octahydrophenanthrene compounds of the general formula I is stated in Table 1 and serves as a measure of the affinity of the compounds of the general formula I according to the invention with the (+)-MK801 binding site of the ionotropic NMDA receptor.

Affinity with the (+)-MK801 binding site of the ionotropic NMDA-receptor was determined for each of these compounds (1) to (6) in accordance with the stated molecular pharmacological investigations. The corresponding percentage inhibition values of specific binding of the ligand (³H)-(+)-MK801 are stated in the Table below.

TABLE 1

| | Percentage inhibition of |
|---|---|
| Compound no. | (+)-MK801 binding at 10 μM |
| 1 | 46 |
| 2 | 56 |
| 3 | 40 |
| 4 | 36 |
| 5 | 28 |
| 6 | 31 |

Pharmacological Investigations:

Writhing Test in Mice

The analgesic efficacy of the compounds according to the invention was investigated by phenylquinone-induced writhing in mice, modified after I.C. Hendershot, J. Forsaith in J. Pharmacol. Exp. There. 125, 237-240 (1959). Male mice weighing 25-30 g were used for this purpose. Groups of 10 animals per substance dose received, 1.0 minute after intravenous administration of the substances tested, 0.3 ml/mouse of a 0.02% aqueous solution of phenylquinone (phenylbenzoquinone, Sigma, Deisenhofen; solution prepared with addition of 5% of ethanol and stored in a water bath at 45° C.) administered intraperitoneally. The animals were then placed individually in observation cages. A push button counter was used to record the number of pain-induced stretching movements (writhing reactions=straightening of the torso with stretching of the rear extremities) for 5-20 minutes after phenylquinone administration. The control was provided by animals who received only physiological common salt solution with phenylquinone.

All the substances were tested at the standard dosage of 10 mg/kg. The percentage inhibition (% inhibition) of the writhing reactions by a substance was calculated according to the following formula:

$$\% Inhibition = 100 - [ \frac{Writhing reaction , treated animals}{Writhing reaction , control} \times 100 ]$$

All the investigated compounds according to the invention exhibited a moderately strong to strong analgesic action.

The assignment of the compounds in Table 2 is identical to the assignment in Table 1 above.

TABLE 2

| | Percentage inhibition of writhing |
|---|---|
| Compound no. | reaction at 10 mg/kg i.v. |
| 1 | 74 |
| 2 | 75 |
| 3 | 85 |

1. Substituted octahydrophenanthrene compounds of the general formula I 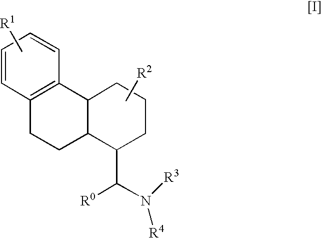
in which
R⁰, R¹ and R², identical or different, denote hydrogen, a linear or branched, saturated or unsaturated C₁-C₁₂ aliphatic residue, a cycloaliphatic saturated or unsaturated C₃-C₇ residue, an aryl or heteroaryl residue optionally attached via a C₁-C₃ alkylene residue, a halogen or a group of the formula —CN, —OR⁵, —SR⁵, —CHF₂, —CF₃, —NHR⁵, —N(R⁵)₂, —NO₂, —SO₂R⁵, or R² denotes an oxo residue,
R³ and R⁴, identical or different, denote hydrogen, a linear or branched, saturated or unsaturated C₁-C₁₂ aliphatic residue, a cycloaliphatic saturated or unsaturated C₃-C₇ residue, an aryl or heteroaryl residue optionally attached via a C₁-C₃ alkylene residue or R³ and R⁴ together form a (CH₂)₂₋₇ ring and
R⁵ denotes a linear or branched, saturated or unsaturated C₁-C₁₂ aliphatic or a C₃-C₇ cycloaliphatic residue, an aryl or heteroaryl residue,
in the form of the racemates, diastereomers or enantiomers thereof and in the form of corresponding bases or of a corresponding physiologically acceptable salt.

2. Substituted octahydrophenanthrene compounds according to claim 1, characterised in that R¹ denotes hydrogen, a C₁-C₆ alkyl residue, a methoxy, hydroxyl, benzyl, phenethyl residue or a halogen, preferably chlorine or fluorine.

3. Substituted octahydrophenanthrene compounds according to claim 1 or 2, characterised in that R² denotes a C₁-C₆ alkyl residue, a benzyl or phenethyl residue, preferably a tert-butyl residue.

4. Substituted octahydrophenanthrene compounds according to one of claims 1 to 3, characterised in that R³ and/or R⁴ denote a C₁-C₃ alkyl residue, each preferably denoting a methyl residue.

5. Substituted octahydrophenanthrene compounds according to one of claims 1 to 4, characterised in that R⁵ denotes a C₁-C₃ alkyl residue, preferably a methyl residue.

6. A substituted octahydrophenanthrene compound from the group comprising:
8-dimethylaminomethyl-4b,5,6,7,8,8a,9,10-octahydrophenanthrene,
8-dimethylaminomethyl-3-methoxy-4b,5,6,7,8,8a,9,10-octahydrophenanthrene,
8-dimethylaminomethyl-4b,5,6,7,8,8a,9,10-octahydrophenanthren-3-ol,
8-dimethylaminomethyl-2-fluoro-4b,5,6,7,8,8a,9,10-octahydrophenanthrene,
8-dimethylaminomethyl-3-chloro-4b,5,6,7,8,8a,9,10-octahydrophenanthrene,
6-tert-butyl-8-dimethylaminomethyl-4b,5,6,7,8,8a,9,10-octahydrophenanthrene,
and a corresponding physiologically acceptable salt, preferably a hydrochloride.

7. A process for the production of substituted octahydrophenanthrene compounds according to one of claims 1 to 6, characterised in that
a compound of the general formula II is converted, 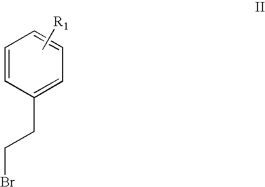
with Mg in a suitable organic solvent, preferably in an organic solvent containing ether, particularly preferably in diethyl ether or tetrahydrofuran, and optionally under a protective gas atmosphere, preferably under argon gas, into the corresponding Grignard compound, then the reaction mixture is cooled, preferably to 10 to 30° C., and this reaction mixture is reacted with a compound of the general formula III, 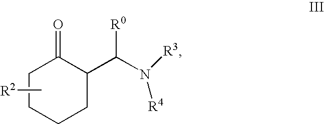
which is optionally dissolved in an organic solvent and the resultant compound IV 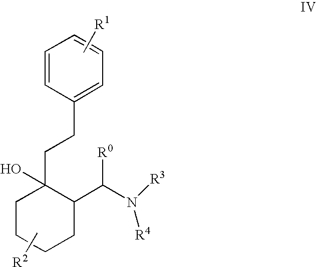
is optionally purified and/or isolated using conventional methods,
the compound IV is reacted by heating with a suitable acid, preferably HBr or formic acid, in a suitable solvent, preferably water or formic acid, is neutralised with a suitable base and is optionally purified and/or isolated using conventional methods,
in which the residues R⁰-R⁵ have the meaning according to claim 1.

8. A process according to claim 7, characterised in that the reaction of the compounds of the general formulae II and III to yield a compound of the general formula IV proceeds within 0.1 to 5 hours, preferably 0.5 to 2 hours.

9. A process according to claim 7 or 8, characterised in that the reaction of the compound of the general formula IV with a suitable acid proceeds by heating to 50 to 150° C., preferably to 80 to 120° C.

10. A process according to claims 7 to 9, characterised in that the reaction of the compound of the general formula IV with a suitable acid proceeds over 1 to 8 hours, preferably for 3 to 6 hours.

11. A process for the production of the hydrochloride of a compound according to one of claims 1 to 6, characterised in that a compound of the general formula I is reacted, optionally in a suitable solvent, preferably ethyl methyl ketone, with trimethylchlorosilane.

12. A process according to claim 11, characterised in that the reaction proceeds, optionally with stirring, over a period of 2 to 8 hours, preferably over 3 to 6 hours.

13. A process according to claim 11 or 12, characterised in that the reaction proceeds, optionally with stirring, at 0 to 50° C., preferably at 20 to 30° C.

14. A pharmaceutical preparation containing at least one substituted octahydrophenanthrene compound according to one of claims 1 to 6 and optionally physiologically acceptable auxiliary substances.

15. A pharmaceutical preparation according to claim 14 at least containing a mixture of the enantiomers of a compound according to one of claims 1 to 6, wherein the two enantiomers are present in non-equimolar quantities.

16. A pharmaceutical preparation according to claim 14 at least containing a mixture of the enantiomers of a compound according to one of claims 1 to 6, wherein one of the enantiomers is present in a relative proportion of between 5 and 45 weight percent in the enantiomer mixture.

17. A pharmaceutical preparation according to claim 14 for combatting pain.

18. A pharmaceutical preparation according to claim 17 for combatting chronic pain.

19. A pharmaceutical preparation according to claim 17 for combatting neuropathic pain.

20. A pharmaceutical preparation according to claim 14 for the treatment or prevention of neurodegenerative diseases, preferably of Alzheimer's disease, Parkinson's disease or Huntington's chorea.

21. A pharmaceutical preparation according to claim 14 for the treatment or prevention of stroke.

22. A pharmaceutical preparation according to claim 14 for the treatment or prevention of cerebral ischaemia.

23. A pharmaceutical preparation according to claim 14 for the treatment or prevention of cerebral infarct.

24. A pharmaceutical preparation according to claim 14 for the treatment or prevention of cerebral oedema.

25. A pharmaceutical preparation according to claim 14 for anxiolysis.

26. A pharmaceutical preparation according to claim 14 for anaesthesia.

27. A pharmaceutical preparation according to claim 14 for the treatment or prevention of schizophrenia.

28. A pharmaceutical preparation according to claim 14 for the treatment or prevention of psychoses brought about by elevated amino acid levels.

29. A pharmaceutical preparation according to claim 14 for the treatment or prevention of AIDS dementia.

30. A pharmaceutical preparation according to claim 14 for the treatment or prevention of Tourette's syndrome.

31. A pharmaceutical preparation according to claim 14 for the treatment or prevention of inflammatory and/or allergic reactions.

32. A pharmaceutical preparation according to claim 14 for the treatment or prevention of insufficiency states of the central nervous system, preferably hypoxia or anoxia.

33. A pharmaceutical preparation according to claim 14 for the treatment or prevention of perinatal asphyxia.

34. A pharmaceutical preparation according to claim 14 for the treatment or prevention of depression.

35. A pharmaceutical preparation according to claim 14 for the treatment or prevention of mental health conditions.

36. A pharmaceutical preparation according to claim 14 for the treatment or prevention of epilepsy.

37. A pharmaceutical preparation according to claim 14 for the treatment or prevention of urinary incontinence.

38. A pharmaceutical preparation according to claim 14 for the treatment or prevention of pruritus.

39. A pharmaceutical preparation according to claim 14 for the treatment or prevention of tinnitus.

40. A pharmaceutical preparation according to claim 14 for the treatment or prevention of diarrhoea.

41. Use of at least one substituted octahydrophenanthrene according to one of claims 1 to 6 for the production of a pharmaceutical preparation for combatting pain, preferably chronic or neuropathic pain.

42. Use of at least one substituted octahydrophenanthrene according to one of claims 1 to 6 for the production of a pharmaceutical preparation for the treatment or prevention of neurodegenerative diseases, preferably of Alzheimer's disease, Parkinson's disease or Huntington's chorea, for the treatment or prevention of migraine, stroke, cerebral ischaemia, cerebral infarct, cerebral oedema, schizophrenia, psychoses brought about by elevated amino acid levels, AIDS dementia, Tourette's syndrome, inflammatory and/or allergic reactions, insufficiency states of the central nervous system, in particular hypoxia and anoxia, perinatal asphyxia, depression, mental health conditions, epilepsy, urinary incontinence, pruritus, tinnitus, diarrhoea, for anxiolysis or for anaesthesia.
